(19) United States
(12) Patent Application Publication
(10) Pub. No.: US 2013/0102565 A1
(43) Pub. Date: Apr. 25, 2013

(54) SEMULOPARIN FOR THE PREVENTION OF A MORTALITY AND/OR MORBIDITY EVENT IN A PATIENT UNDERGOING MAJOR ORTHOPEDIC SURGERY

(71) Applicant: AVENTIS PHARMA S.A., Paris (FR)

(72) Inventors: Dominique DESTREE, Paris (FR); Chantal JEAN DE POTTER, Paris (FR); Francesca LAWSON, Paris (FR); Christelle LORENZATO, Paris (FR)

(73) Assignee: AVENTIS PHARMA S.A., Paris (FR)

(21) Appl. No.: 13/712,424

(22) Filed: Dec. 12, 2012

Related U.S. Application Data
(63) Continuation of application No. PCT/EP2011/060597, filed on Jun. 24, 2011.

(30) Foreign Application Priority Data
Jun. 25, 2010 (EP) 10305683.4

(51) Int. Cl. A61K 31/727

(52) U.S. Cl. CPC A61K 31/727
USPC 514/56

(57) ABSTRACT

The invention relates to the use of semuloparin or a pharmaceutically acceptable salt thereof for the prevention of a mortality and/or morbidity event, more specifically venous thromboembolism and death, in a patient undergoing major orthopedic surgery, such as hip replacement, knee replacement or hip fracture surgery.

The invention relates to the use of semuloparin or a pharmaceutically acceptable salt thereof for the prevention of a mortality and/or morbidity event, more specifically venous thromboembolism and death, in a patient undergoing major orthopedic surgery.

Semuloparin, or AVE5026 (sanofi-aventis laboratory code) belongs to a new generation of hemisynthetic heparins. It is a new ultra-low molecular weight heparin, with an average molecular weight of 2000-3000 Daltons and a novel antithrombotic profile resulting from high anti-Factor Xa activity (between 145 and 180 U/mg, average value of ˜160 U/mg) and residual anti-Factor IIa activity (less than 5 U/mg, on average ˜2 U/mg). It is obtained by selective and controlled depolymerization of heparin by a phosphazene base, as described for example in Journal of Thrombosis and Haemostasis, 2009, vol. 7, 1143-1151, as well as in the patent applications WO 02/08295 and in particular in WO 2004/033503. Semuloparin, in the form of its sodium salt, is in clinical development for venous thromboembolism prevention.

At the present time, low-molecular-weight heparins (LMWHs), the synthetic pentasaccharide fondaparinux or dose-adjusted anti-vitamin K are the primary treatments for the prevention of venous thromboembolic diseases.

Patients undergoing surgery are at substantial risk of postoperative venous thromboembolism (hereafter “VTE”). In addition, many hospitalized patients have additional risk factors for VTE. In order to avoid postoperative venous thromboembolic complication, guidelines in thrombosis (ACCP guidelines) recommend the use of antithrombotic drugs for certain categories of surgical patients. Amongst these drugs, the LMWH enoxaparin is the pharmacological VTE prevention agent with the highest clinical documentation in surgical populations and with the largest clinical use in this setting. Enoxaparin has an average molecular weight of 3800-5000 Daltons, an anti-Factor Xa activity comprised between 90 and 125 IU/mg and an anti-Factor IIa activity of 20-35 IU/mg.

The Applicant has now found that a product outside of the LMWH class, namely the ultra-low molecular weight heparin (ULMWH) semuloparin, allows to reduce the occurrence of mortality and/or morbidity events in patients undergoing major orthopedic surgery.

Therefore, the subject-matter of the invention is an ultra-low molecular weight heparin (ULMWH) with an average molecular weight of 2000 to 3000 Daltons, an anti-Factor Xa (anti-FXa) activity of 145 to 180 U/mg and an anti-Factor IIa (anti-FIIa) activity of less than 5 U/mg, for use in the prevention of a mortality and/or morbidity event in a patient undergoing major orthopedic surgery, wherein said event is selected from VTE and death and wherein the efficacy of said use is clinically proven by phase III clinical trials.

The anti-FXa and anti-FIIa activities described above are measured using amidolytic methods on a chromogenic substrate as adapted from the monograph on LMWHs of the European Pharmacopeia in force, using as reconstitution buffer a tris-NaCl pH 7.4 buffer comprising PEG6000 (polyethylene glycol 6000) instead of albumin, and an ULMWH reference substance with an anti-FXa activity of 159 U/mg and an anti-FIIa activity of 2.9 U/mg. The potencies are expressed in units per mg due to the use of an internal ULMWH reference standard. Indeed, as a function of the concentration/dilution, lack of parallelism can be observed for anti-FIIa activities routine determination when the ULMWH is calibrated versus LMWH standard. The anti-FXa and anti-FIIa activities of the ULMWH reference substance described above have been determined relative to the international LMWH standard on a range of dilution where the parallelism was obtained. The results of the dosages are exploited according to §5.3 of the European Pharmacopeia in force (“Statistical analyses of dosages and biological assays results”).

More particularly, the above ULMWH is semuloparin and the subject-matter of the invention is therefore semuloparin for use in the prevention of a mortality and/or morbidity event in a patient undergoing major orthopedic surgery, wherein said event is selected from VTE and death and wherein the efficacy of said use is clinically proven by phase III clinical trials.

The term “semuloparin”, in the framework of the instant invention, encompasses any pharmaceutically acceptable salt thereof, in particular its sodium salt. The term “semuloparin” shall therefore be understood herein as “semuloparin or any pharmaceutically acceptable salt thereof”.

According to the present invention, the terms below have the following meanings:
“major orthopedic surgery” refers to hip replacement, hip fracture or knee replacement surgery;
“a patient” refers to a patient being at risk of VTE and for whom prophylaxis of venous thromboembolic events is indicated;
“death” refer to all causes of death;
“phase III clinical trial” refers to a multicenter, randomized, double-blinded study involving a large patients group (1.000 to more than 2.000), aiming at being the definitive assessment of how effective the drug is, in comparison with current standard treatment.

More specifically, the subject-matter of the invention is the above-defined ULMWH for use in the prevention of a mortality and/or morbidity event in a patient undergoing major orthopedic surgery, being at risk of VTE and for whom prophylaxis of venous thromboembolic events is indicated, wherein said mortality and/or morbidity event is selected from VTE and death and wherein the efficacy of said use is clinically proven by phase III clinical trials.

In particular, the subject-matter of the invention is said ULMWH for its use as described above, wherein said mortality and/or morbidity event is selected from deep vein thrombosis (hereafter “DVT”, including proximal or distal DVT), non-fatal pulmonary embolism and death.

In the framework of the present invention, the terms below have the following meanings:
“deep vein thrombosis”: designates a blood clot in a deep vein of the lower limbs;
“proximal DVT”: designates a DVT event occurring above the knee;
“distal DVT”: designates a DVT event occurring below the knee.

In another aspect of the instant invention, major orthopedic surgery is selected from hip replacement surgery, hip fracture surgery or knee replacement surgery. More particularly, major orthopedic surgery is selected from hip surgery, including hip replacement and hip fracture surgery.

In another embodiment, the invention relates to said ULMWH for use in the prevention of a mortality and/or morbidity event in a patient undergoing major orthopedic surgery, wherein said event is selected from venous thromboembolism and death, wherein the efficacy of said use is clinically proven by phase III clinical trials, and wherein said ULMWH displays a better efficacy compared to a standard antithrombotic treatment.

In particular, said standard antithrombotic treatment is the LMWH enoxaparin.

Indeed, it has been found that administration of the above-defined ULMWH after major orthopedic surgery enables to decrease the occurrence of venous thromboembolism and death in the patients having benefited from a treatment with said ULMWH, compared to the occurrence of such events in patients having benefited from the standard treatment with enoxaparin. “Treatment” is understood herein as a venous thromboprophylactic treatment.

In this embodiment of the invention, said mortality and/or morbidity event is in particular deep vein thrombosis.

In this embodiment of the invention, said patient is in particular undergoing hip replacement surgery.

In another embodiment, the invention relates to a method for the prevention of a mortality and/or morbidity event in a patient undergoing major orthopedic surgery, which comprises the administration of an effective dose of the above-defined ULMWH to a patient in need thereof, wherein said event is selected from venous thromboembolism and death and wherein the efficacy of said use is clinically proven by phase III clinical trials, as described above.

According to the instant invention, the above-defined ULMWH is administered at a 20 mg daily dose to patients with normal renal function or to patients with mild or moderate renal impairment. For patients with severe renal impairment, said ULMWH is administered at a 10 mg daily dose.

According to the instant invention, the renal function of the patients is defined according to estimated creatinine clearance (CLcr) values calculated using the well-known Cockroft-Gault formula, and is classified according to the following characteristics:
normal renal function: CLcr>80 mL/min;
mild renal impairment: 50≦CLcr≦80 mL/min;
moderate renal impairment: 30≦CLcr<50 mL/min;
severe renal impairment: CLcr<30 mL/min.

The treatment with the above-defined ULMWH is advantageously administered once daily. As used therein, “daily” means an administration every 24 hours plus or minus 4 hours. Said treatment is advantageously administered for 7 to 10 days.

As used herein, the wording “ULMWH for use in . . . ” shall be understood as being equivalent to the wording “use of the ULMWH for . . . ” or “use of the ULMWH for the preparation of a medicament for use in . . . ”.

The invention therefore also relates to the use of the ULMWH defined above for the manufacture of a medicament useful for the prevention of a mortality and/or morbidity event in a patient undergoing major orthopedic surgery, wherein said event is selected from venous thromboembolism and death and wherein the efficacy of said use is clinically proven by phase III clinical trials.

The invention also relates to an article of manufacture comprising:
a packaging material,
a compound chosen from an ULMWH with an average molecular weight of 2000 to 3000 Daltons, an anti-FXa activity of 145 to 180 U/mg and an anti-FIIa activity of less than 5 U/mg, in particular semuloparin or a pharmaceutically acceptable salt thereof, and
a label or package insert contained within said packaging material indicating that said compound is effective for the prevention of venous thromboembolism (VTE) and death in patients undergoing major orthopedic surgery.

The embodiments of the invention described above also apply to this article of manufacture.

Having now described the present invention, the same will be more clearly understood by reference to the following examples of the invention, which are included herewith for purposes of illustration only and are not intended to be limiting of the invention.

The following abbreviations shall be used:

b.i.d.: bis in die (twice daily)

CLcr: Creatinine Clearance

DVT: deep vein thrombosis

IP: investigational product

PE: pulmonary embolism

UFH: unfractionated heparin

LMWH: low molecular weight heparin

OR: Odds Ratio

q.d.: quaque die (once daily)

s.c.: subcutaneously

SRI: severe renal impairment

VTE: venous thromboembolism

95% exact CI: 95% exact Confidence Interval

95% mid-p CI: 95% mid-p Confidence Interval

### EXAMPLE 1

### Preparation of Semuloparin

Semuloparin, in the form of a sodium salt, is obtained by a chemoselective depolymerization of heparin, activated through its benzyl ester derivative, by the phosphazene base 2-tert-butylimino-2-diethylamino-1,3-dimethylperhydro-1,2,3-diazaphosphorine (BEMP). The hemisynthetic pathway, allowing to recover semuloparin in the form of a sodium salt, is described in the patent applications WO 02/08295 and WO 2004/033503, and in Journal of Thrombosis and Haemostasis, 2009, vol. 7, 1143-1151. This procedure yields semuloparin with an average molecular weight of 2000-3000 Daltons (around 2400 Da on average), an anti-Factor Xa activity of between 145 and 180 U/mg (average value ˜160 U/mg) and a residual anti-Factor IIa activity of less than 5 U/mg (on average ˜2 U/mg).

### EXAMPLE 2

### The SAVE-KNEE Study

A Multinational, Multicenter, Randomized, Double Blind Study comparing the Efficacy and Safety of AVE5026 with enoxaparin for the Prevention of Venous Thromboembolism in Patients Undergoing Elective Knee Replacement Surgery.

1) Study Objectives

The primary objective of the study is to compare the efficacy of once daily (q.d.) subcutaneous (s.c.) injections of 20 mg AVE5026 (10 mg for SR1 patients) with twice daily (b.i.d.) s.c. injections of 30 mg enoxaparin (20 mg q.d. for SR1 patients) administered during 7-10 days after surgery for the prevention of venous thromboembolic events in patients undergoing elective knee replacement surgery. The secondary objectives of this study are to evaluate the safety of AVE5026 in patients undergoing elective knee replacement surgery and to document AVE5026 exposures in this population.

2) Study Design

Patient's eligibility is determined during the screening period (within the 2 weeks prior to surgery) and is reviewed before randomization.

Randomized treatment is allocated to eligible patients, taking into account the geographical region of the patient and the estimated CLcr at baseline (< or ≧30 mL/min).

An end of treatment visit is performed the day of last IP injection or at Day 10, whichever comes first. A bilateral venography is performed between Day 7 and Day 11. A follow-up visit is scheduled at Day 35-42. Maximum duration of study participation is therefore 42 days, including a treatment period up to Day 7-10 and a follow-up period with a visit at Day 35-42.

3) Patients

A total of 1150 patients are randomized in the study.

Patients meeting the following criteria are suitable for enrolment in the study:
Elective knee replacement surgery or a revision of at least one component of a previously implanted knee prosthesis performed 6 months prior to study entry.
Signed written informed consent.

Patients meeting one of the following criteria are excluded from enrolment into the study:

1. Legal lower age limitations (country specific).

2. Any major orthopedic surgery in the 3 months prior to study start.

3. Elective knee surgery with polyethylene liner exchange only.

4. First step of a two-step exchange arthroplasty for infection after knee replacement.

5. Hemostasis prior to the first IP injection not established

6. Clinical signs or symptoms of DVT or PE within the last 12 months or known post phlebitic syndrome.

7. Known sensitivity to iodine or contrast dyes, and any contra-indications to the performance of venography.

8. Any treatment or procedure within 2 weeks prior to randomization, or planned during the course of the study treatment period, that could affect the incidence of VTE, such as:
Parenteral anticoagulants (UFH, LMWH [e.g., enoxaparin, dalteparin, nadroparin], fondaparinux, bivalirudin, hirudin)
Oral anticoagulants (vitamin K antagonists)
GPIIb/IIIa antagonists: abciximab, eptifibatide, tirofiban
Thrombolytic agents
Dextrans
Intermittent pneumatic compression of the legs (IPC)

9. Known progressive malignant disease.

10. Subject unlikely to comply with protocol (e.g., uncooperative attitude, inability to return for follow-up visits, inability to receive daily injection by a Health Care Professional after hospital discharge and unlikelihood of completing the study).

11. Treatment with any investigational product or investigational device in the last 30 days or 5 half lives (whichever is longer) prior to randomization.

12. Any previous exposure to AVE5026

13. Active major bleeding.

14. Thrombocytopenia associated with a positive in vitro test for anti-platelet antibody in the presence of enoxaparin sodium.

15. Known hypersensitivity to enoxaparin sodium (e.g., pruritus, urticaria, anaphylactoid reactions).

16. Known hypersensitivity to heparin or pork products.

17. Conditions with increased risk of hemorrhage, such as bacterial endocarditis, congenital or acquired bleeding disorders, active ulcerative and angiodysplastic gastrointestinal disease, hemorrhagic stroke, or shortly after brain, spinal, or ophthalmological surgery.

18. End stage renal disease (estimated creatinine clearance <10 mL/min) or patient on dialysis.

19. Pregnant or breast-feeding women.

20. Women of childbearing potential not protected by highly effective contraceptive method of birth control as defined for contraception in the Informed Consent Form for the duration of the study and/or who are unwilling or unable to be tested for pregnancy.

4) Treatments

Sanofi-aventis supplies and manufactures the blinded treatments for this study. According to their randomized assignments, patients receive either AVE5026 or enoxaparin. Both treatments are presented as a ready-to-use 0.5 ml prefilled syringe, identical in appearance, and containing the same volume of a sterile, isotonic solution with sodium chloride 0.9% and water for injection.

The matching placebo syringe is strictly identical in appearance, containing the same volume but without active component.

AVE5026 and enoxaparin are administered subcutaneously. The entire volume of the pre-filled syringe must be injected.

Investigational Product (IP) is administered in a blind manner for 7 to 10 days after surgery (Day 1 is the day of surgery). Patients receive either:
Enoxaparin, 30 mg sc b.i.d. (20 mg q.d. for patients with SRI, defined as estimated CLcr<30 mL/min), or
AVE5026, 20 mg sc q.d. (10 mg q.d. for patients with SRI).

5) Results

The efficacy analysis period is defined as the period from randomization up to Day 11 (Day 1 being the day of surgery) or up to the mandatory bilateral venography, whichever comes first.

The primary efficacy population includes all randomized patients who received at least one IP injection (active or placebo), who underwent elective knee replacement surgery and with a non-missing primary efficacy endpoint.

The primary analysis compares the two groups (semuloparin and enoxaparin) using an exact 2-sided stratified test at a a level of 0.05 (Gart 1970). Event rates per treatment group are summarized.

Tables 1 and 2 describe the primary efficacy analysis of the SAVE-KNEE study.

TABLE 1
Any VTE or death during the efficacy analysis period

| | Semuloparin | Enoxaparin |
|---|---|---|
| | (N = 428) | (N = 427) |
| Any VTE or death: | | |
| n (%) | 105 (24.5%) | 120 (28.1%) |
| Comparison versus enoxaparin: | | |
| Common OR (95% exact Cl) | | 0.83 (0.60 to 1.14) |
| p-value | | 0.2440 |
| Relative risk (95% Cl) | | 0.87 (0.70 to 1.09) |

N: number of patients in the primary efficacy population
n: number of patients showing a given event

TABLE 2
Components of the primary efficacy endpoint

| | | | OR (95% | Relative |
|---|---|---|---|---|
| | | | mid-p | risk |
| | Semuloparin | Enoxaparin | Cl) | (95% Cl) |
| Any DVT | | | 0.83 | 0.87 |
| n/N (%) | 105/428 (24.5%) | 120/427 (28.1%) | (0.61 | (0.70 |
| | | | to 1.13) | to 1.09) |
| Any proximal | | | 1.74 | 1.71 |
| DVT | 17/483 (3.5%) | 10/487 (2.1%) | (0.79 | (0.79 |
| n/N (%) | | | to 3.99) | to 3.71) |
| Distal DVT | | | 0.77 | 0.81 |
| only | 84/425 (19.8%) | 103/424 (24.3%) | (0.55 | (0.63 |
| n/N (%) | | | to 1.06) | to 1.05) |
| Non-fatal | | | 0.00 | NA |
| PE | 0/573 (0%) | 1/568 (0.2%) | (0.00 | |
| n/N (%) | | | to 18.83) | |
| All cause | 0/573 (0%) | 0/568 (0%) | NA | NA |
| death | | | | |
| n/N (%) | | | | |

N: number of efficacy evaluable patients
n: number of patients showing a given event
NA: non applicable

These results suggest that semuloparin has similar efficacy as enoxaparin for the prevention of VTE and death (DVT, non-fatal PE and death) in patients undergoing elective knee replacement surgery.

As apparent from table 1, the ULMWH involves a 13% relative risk reduction in the occurrence of VTE or death, compared to a treatment with enoxaparin.

### EXAMPLE 3

The SAVE-HIP1 Study

A Multinational, Multicenter, Randomized, Double Blind Study comparing the Efficacy and Safety of AVE5026 with enoxaparin for the Prevention of Venous Thromboembolism in Patients Undergoing Elective Total Hip Replacement Surgery.

1) Study Objectives

The primary objective of the study is to compare the efficacy of once daily (q.d.) subcutaneous (s.c.) injections of 20 mg AVE5026 (10 mg for patients with SR1) with q.d. s.c. injections of 40 mg enoxaparin (20 mg for patients with SR1) administered during 7-10 days after surgery for the prevention of venous thromboembolic events in patients undergoing elective hip replacement surgery. The secondary objectives of this study are to evaluate the safety of AVE5026 in patients undergoing elective total hip replacement surgery and to document AVE5026 exposures in this population.

2) Study Design

Patient's eligibility is determined during the screening period (within the 2 weeks prior to surgery) and is reviewed before randomization (day before surgery or day of surgery).

Randomized treatment is allocated to eligible patients, taking into account the geographical region of the patient, the timing of the first IP injection and the estimated CLcr at screening (< or ≧30 mL/min).

An end of treatment visit is performed the day of last IP injection or at Day 10, whichever comes first. A bilateral venography is performed between Day 7 and Day 11. A follow-up visit is scheduled at Day 35-42.

Maximum duration of study participation is therefore 42 days, including a treatment period up to Day 7-10 and a follow-up period with a visit at Day 35-42.

3) Patients

A total of 2326 patients are randomized in the study.

Patients meeting the following criteria are suitable for enrolment in the study:
Elective hip replacement surgery or a revision of at least one component of a previously implanted total hip prosthesis performed 6 months prior to study entry;
Signed written informed consent.

Patients meeting one of the following criteria are excluded from enrolment into the study:

1. Elective hip surgery with polyethylene liner exchange only.

2. First step of a two-step exchange arthroplasty for infection after hip prosthesis replacement.

3. Any of the exclusion criteria numbered as items 1, 2 and 6-20 under example 2 above (SAVE-KNEE study).

4) Treatments

Sanofi-aventis supplies and manufactures the blinded treatments for this study. According to their randomized assignments, patients receive either AVE5026 or enoxaparin. Both treatments are presented as a ready-to-use 0.5 ml prefilled syringe, identical in appearance, and containing the same volume of a sterile, isotonic solution with sodium chloride 0.9% and water for injection.

Regarding the pre-operative IP injection (if planned) and the post-operative IP injections to be administered 8±1 hours and 12±1 hours after the end of the surgery, the corresponding pre-filled syringes contain either active drug or placebo.

The matching placebo syringe is strictly identical in appearance, containing the same volume but without active component.

AVE5026 or enoxaparin are administered subcutaneously. The entire volume of the pre-filled syringe must be injected.

Investigational Product (IP) is administered in a blinded manner once daily during 7-10 days after surgery. Patients receive either enoxaparin, or AVE5026. The first pre-operative injection (enoxaparin for patients allocated to comparator group and placebo for patients allocated to AVE5026 group) is administered 12±1 hours before the surgical procedure. The pre-operative injection may be omitted as per local enoxaparin labeling. The first post-operative injection (AVE5026 or placebo) is administered 8±1 hours after incision closure, provided that hemostasis has been established. The second post-operative injection (enoxaparin or placebo) is administered 12±1 hours after incision closure. Then, in any case, patients receive on the following days once daily IP injection (enoxaparin or AVE5026).

In case of no pre-operative injection, the first post-operative injection (AVE5026 or placebo) is administered 8±1 hours after incision closure on Day 1, provided that hemostasis has been established. The second post-operative injection (enoxaparin or placebo) is administered 12±1 hours after incision closure. Then, one daily injection (AVE5026 or enoxaparin) is administered on the following days, in the morning from Day 2 and for 7 to 10 days.

5) Results

The efficacy analysis period is defined as the period from randomization up to Day 11 (Day 1 being the day of surgery) or up to the mandatory bilateral venography, whichever comes first.

The primary efficacy population includes all randomized patients who received at least one IP injection (active or placebo), who underwent elective hip replacement surgery and with a non-missing primary efficacy endpoint.

The primary analysis compares the two groups (semuloparin and enoxaparin) using an exact 2-sided stratified test at a level of 0.05 (Gart 1970). Event rates per treatment group are summarized.

Tables 3 and 4 describe the primary efficacy analysis of the SAVE-HIP1 study.

TABLE 3
Any VTE or death during the efficacy analysis period

| | Semuloparin | Enoxaparin |
|---|---|---|
| | (N = 916) | (N = 933) |
| Any VTE or death: | | |
| n (%) | 58 (6.3%) | 104 (11.1%) |
| Comparison versus enoxaparin: | | |
| Common OR (95% exact Cl) | | 0.54 (0.38 to 0.76) |
| p-value | | 0.0003 |
| Relative risk (95% Cl) | | 0.57 (0.42 to 0.77) |

N: number of patients in the primary efficacy population
n: number of patients showing a given event

TABLE 4
Components of the primary efficacy endpoint

| | | | OR | Rel- |
|---|---|---|---|---|
| | | | (95% | ative |
| | | | mid-p | risk |
| | Semuloparin | Enoxaparin | Cl) | (95% Cl) |
| Any DVT | | | 0.54 | 0.57 |
| n/N (%) | 57/915 (6.2%) | 102/931 (11.0%) | (0.38 | (0.42 |
| | | | to 0.76) | to 0.78) |
| Any proximal | | | 0.87 | 0.87 |
| DVT | 13/1002 (1.3%) | 15/1011 (1.5%) | (0.40 | (0.42 |
| n/N (%) | | | to 1.86) | to 1.83) |
| Distal DVT | | | 0.49 | 0.51 |
| only | 42/915 (4.6%) | 83/930 (8.9%) | (0.33 | (0.36 |
| n/N (%) | | | to 0.72) | to 0.74) |
| Non-fatal | 0/1150 (0%) | 0/1152 (0%) | NA | NA |
| PE | | | | |
| n/N (%) | | | | |
| All cause | | | 0.50 | 0.50 |
| death | 1/1150 (<0.1%) | 2/1152 (0.2%) | (0.02 | (0.05 to |
| n/N (%) | | | to 6.59) | 5.52) |

N: number of efficacy evaluable patients
n: number of patients showing a given event
NA: non applicable

These results demonstrate that semuloparin is effective in preventing VTE and death (DVT, non-fatal PE and death) in patients undergoing elective total hip replacement surgery.

These results further demonstrate that semuloparin displays better efficacy than enoxaparin for the prevention of VTE and death, in particular for the prevention of DVT (more specifically distal DVT) in those patients.

As apparent from tables 3 and 4, the ULMWH involves a 43% relative risk reduction in the occurrence of VTE or death compared to a treatment with enoxaparin, while the relative risk reduction in the occurrence of DVT is 43% (13% for proximal DVT and 49% for distal DVT).

### EXAMPLE 4

The SAVE-HIP2 study. A Multinational, Multicenter, Randomized, Double Blind Study comparing the Efficacy and Safety of AVE5026 with enoxaparin for the Prevention of Venous Thromboembolism in Patients Undergoing Hip Fracture Surgery.

1) Study Objectives

The primary objective of the study is to compare the efficacy of once daily (q.d.) subcutaneous (s.c.) injections of 20 mg AVE5026 (10 mg for patients with SR1) with q.d. s.c. injections of 40 mg enoxaparin (20 mg for patients with SR1) administered for 7-10 days after surgery for the prevention of venous thromboembolic events in patients undergoing hip fracture surgery. The secondary objectives of this study are to evaluate the safety of AVE5026 in patients undergoing hip fracture surgery and to document AVE5026 exposures in this population.

2) Study Design

Patient's eligibility is determined at the admission:
a patient is eligible for a pre-operative randomization if a pre-operative injection of enoxaparin is requested as per local labelling. In this case, this pre-operative IP injection (enoxaparin in the comparator group and placebo in the AVE5026 group) is administered 12±1 hours before start of surgery;
a patient is eligible for a post-operative randomization if the post-operative hemostasis has been established just prior to the first IP injection that will be administered 8±1 hours after the end of surgery.

Randomized treatment is allocated to eligible patients, taking into account the geographical region of the patient, the timing of the first IP injection and the estimated CLcr at screening (< or ≧30 mL/min).

An end of treatment visit is performed the day of last IP injection or at Day 10, whichever comes first. A bilateral venography is performed between Day 7 and Day 11. A follow-up visit is scheduled at Day 35-42.

Maximum duration of study participation is therefore 42 days, including a treatment period up to Day 7-10 and a follow-up period with a visit at Day 35-42 after randomization.

3) Patients

A total of 1003 patients are randomized in the study.

Patients meeting the following criteria are suitable for enrolment in the study:

1. Standard surgery for fracture of the upper third of the femur, including femoral head and neck:
either planned within the first 36 hours* after admission to hospital, and in the 12±1 hours following the pre-operative IP injection,
or just performed 8±1 hours previously (from the time of incision closure), and provided that hemostasis has been established.
   If time from injury to hospital admission is documented to be less than 24 hours, this 36 hours time window can be expanded but in all cases the time from injury to surgery must not exceed 60 hours.

2. Signed written informed consent.

Patients meeting one of the following criteria are excluded from enrolment into the study:

1. Estimated time of injury/fracture >24 hours before admission to hospital.

2. Time from injury/fracture to surgery >60 hours.

3. Multiple trauma affecting more than one organ system.

4. Any of the exclusion criteria numbered as items 1, 2 and 6-20 described under example 2 above (SAVE-KNEE study).

4) Treatments

It is proceeded in the same manner as described in example 3 above (SAVE-HIP1 study).

5) Results

The efficacy analysis period is defined as the period from randomization up to Day 11 (Day 1 being the day of surgery) or up to the mandatory bilateral venography, whichever comes first.

The primary efficacy population includes all randomized patients who received at least one IP injection (active or placebo), who underwent elective hip fracture surgery and with a non-missing primary efficacy endpoint.

The primary analysis compares the two groups (semuloparin and enoxaparin) using an exact 2-sided stratified test at a a level of 0.05 (Gart 1970). Event rates per treatment group are summarized.

Tables 5 and 6 describe the primary efficacy analysis of the SAVE-HIP2 study.

TABLE 5
Any VTE or death during the efficacy analysis period

| | Semuloparin | Enoxaparin |
|---|---|---|
| | (N = 384) | (N = 369) |
| Any VTE or death: | | |
| n (%) | 68 (17.7%) | 81 (22.0%) |
| Comparison versus enoxaparin: | | |
| Common OR (95% exact Cl) | | 0.77 (0.53 to 1.12) |
| p-value | | 0.1699 |
| Relative risk (95% Cl) | | 0.81 (0.60 to 1.08) |

N: number of patients in the primary efficacy population
n: number of patients showing a given event

TABLE 6
Components of the primary efficacy endpoint

| | | | OR (95% | Relative |
|---|---|---|---|---|
| | | | mid-p | risk |
| | Semuloparin | Enoxaparin | Cl) | (95% Cl) |
| Any DVT | | | 0.73 | 0.77 |
| n/N (%) | 63/379 (16.6%) | 79/367 (21.5%) | (0.50 | (0.57 |
| | | | to 1.05) | to 1.04) |
| Any proximal | | | 0.48 | 0.49 |
| DVT | 13/426 (3.1%) | 26/420 (6.2%) | (0.23 | (0.26 |
| n/N (%) | | | to 0.93) | to 0.95) |
| Distal DVT | | | 0.97 | 0.98 |
| only | 49/378 (13.0%) | 48/362 (13.3%) | (0.63 | (0.67 |
| n/N (%) | | | to 1.50) | to 4.42) |
| Non-fatal | 1/488 (0.2%) | 0/499 (0%) | NA | NA |
| PE | | | | |
| n/N (%) | | | | |
| All cause | | | 2.05 | 2.05 |
| death | 4/488 (0.8%) | 2/499 (0.4%) | (0.36 | (0.38 |
| n/N (%) | | | to 16.08) | to 11.11) |

N: number of efficacy evaluable patients
n: number of patients showing a given event
NA: non applicable

These results suggest that semuloparin has similar efficacy as enoxaparin for the prevention of VTE and death (DVT, non-fatal PE and death) in patients having undergone hip fracture surgery. As apparent from table 5, the ULMWH involves a 19% relative risk reduction in the occurrence of VTE or death compared to a treatment with enoxaparin.

### EXAMPLE 5

### Meta-Analysis of the SAVE-KNEE, SAVE-HIP-1 and SAVE-HIP-2 Orthopedic Surgery Results

A meta-analysis of the three studies described in examples 2, 3 and 4 above was performed using a fixed-effect logistic regression model adjusted on study. Of 4479 patients randomized, 3457 (77.2%) were evaluable for the primary efficacy analysis. The occurrence of the primary end-point of any VTE or all-cause death was significantly lower for semuloparin versus enoxaparin, as shown in table 7 below.

TABLE 7
Any VTE or death during the efficacy analysis period

| | Semuloparin | Enoxaparin |
|---|---|---|
| | (N = 1728) | (N = 1729) |
| Any VTE or death: | | |
| n (%) | 231 (13.4%) | 305 (17.6%) |
| Comparison versus enoxaparin: | | |
| Common OR (95% exact Cl) | | 0.70 (0.58 to 0.85) |
| p-value | | 0.0003 |
| Relative Risk (95% Cl) | | 0.75 (0.64 to 0.88) |

N: number of patients in the primary efficacy population
n: number of patients showing a given event

TABLE 8
Componentsof the primary efficacy endpoint

| | | | OR (95% |
|---|---|---|---|
| | | | mid-p |
| | Semuloparin | Enoxaparin | Cl) |
| Any DVT | 225/1722 | 301/1725 | 0.69 (0.57 |
| n/N (%) | (13.1%) | (17.4%) | to 0.84) |
| Any proximal | 43/1912 (2.2%) | 51/1918 (2.7%) | 0.84 (0.55 |
| DVT | | | to 1.27) |
| n/N (%) | | | |
| Distal DVT | 175/1718 (10.2%) | 234/1716 (13.6%) | 0.70 (0.57 |
| only | | | to 0.87) |
| n/N (%) | | | |
| Non-fatal | 1/2211 (<0.1%) | 1/2219 (<0.1%) | 1.01 (0.06 |
| PE | | | to 16.12) |
| n/N (%) | | | |
| All cause | 5/2211 (0.2%) | 4/2219 (0.2%) | 1.27 (0.34 |
| death | | | to 4.75) |
| n/N (%) | | | |

N: number of efficacy evaluable patients
n: number of patients showing a given event

These results demonstrate that semuloparin is effective in preventing VTE and death (DVT, non-fatal PE and death), in patients undergoing major orthopedic surgery. These results further demonstrate the better efficacy of semuloparin compared to enoxaparin for the prevention of VTE and death in this setting.

As apparent from tables 7 and 8, the ULMWH involves a 25% relative risk reduction in the occurrence of VTE or death compared to a treatment with enoxaparin, while the risk reduction in the occurrence of DVT is approximately 31% (˜16% for proximal DVT and ˜30% for distal DVT).

What is claimed is:

1. A method for the prevention of a mortality or morbidity event in a patient undergoing major orthopedic surgery, wherein said event is selected from venous thromboembolism and death, and wherein the efficacy of said use is clinically proven by phase III clinical trials, the method comprising administering an effective dose of an ultra-low molecular weight heparin with an average molecular weight of 2000 to 3000 Daltons, an anti-Factor Xa activity of 145 to 180 U/mg and an anti-Factor IIa activity of less than 5 U/mg.

2. The method according to claim 1, wherein said ultra-low molecular weight heparin is semuloparin or a pharmaceutically acceptable salt thereof.

3. The method according to claim 1, wherein said use results in a relative risk reduction in the occurrence of venous thromboembolism or death of at least 25% compared to a treatment with enoxaparin.

4. The method according to claim 1, for use in the prevention of venous thromboembolism, including deep vein thrombosis, non-fatal pulmonary embolism and death.

5. The method according to claim 4, for use in the prevention of deep vein thrombosis.

6. The method according to claim 5, wherein said use results in a risk reduction in the occurrence of deep vein thrombosis of at least 31% compared to a treatment with enoxaparin.

7. The method according to claim 1, for use in the prevention of proximal deep vein thrombosis.

8. The method according to claim 1, for use in the prevention of distal deep vein thrombosis.

9. The method according to claim 1, wherein said major orthopedic surgery is hip surgery.

10. The method according to claim 9, wherein said major orthopedic surgery is selected from hip fracture surgery and hip replacement surgery.

11. The method according to claim 2, wherein semuloparin displays a better efficacy compared to a standard antithrombotic treatment.

12. The method according to claim 11, wherein said standard antithrombotic treatment is enoxaparin.

13. The method according to claim 11, wherein said mortality or morbidity event is deep vein thrombosis.

14. The method according to claim 11, wherein said mortality or morbidity event is distal deep vein thrombosis.

15. The method according to claim 11, wherein said major orthopedic surgery is hip replacement surgery.

16. The method according to claim 2, wherein semuloparin is administered at a 20 mg daily dose to patients with normal renal function or to patients with mild or moderate renal impairment.

17. The method according to claim 2, wherein semuloparin is administered at a 10 mg daily dose in patients with severe renal impairment.

18. The method according to claim 2, wherein the dose is administered once daily.

19. The method according to claim 2, wherein the dose is administered for 7 to 10 days.

20. An article of manufacture comprising:
a packaging material,
a compound chosen from an ultra-low molecular weight heparin (ULMWH) with an average molecular weight of 2000 to 3000 Daltons, an anti-FXa activity of 145 to 180 U/mg and an anti-FIIa activity of less than 5 U/mg, and
a label or package insert contained within said packaging material indicating that said compound is effective for the prevention of venous thromboembolism and death in patients undergoing major orthopedic surgery.

21. The article according to claim 20 wherein the ULMWH is semuloparin or a pharmaceutically acceptable salt thereof.
